(19) United States
(12) Patent Application Publication
(10) Pub. No.: US 2004/0105880 A1
(43) Pub. Date: Jun. 3, 2004

(54) Interpenetrating polymer network

(76) Inventors: Josephine Sara Turner, Kettleby (CA); Lucie Martineau, Kettleby (CA); Pang Nin Shek, Toronto (CA)

Correspondence Address: George A. Seaby, Seaby & Associates, 603 - 880 Wellingtonn Street, Ottawa, K1R 6K7, CA

(21) Appl. No.: 10/717,528

(22) Filed: Nov. 21, 2003

Related U.S. Application Data
(60) Provisional application No. 60/427,933, filed on Nov. 21, 2002.

(51) Int. Cl. D01D 5/30; A61K 9/70

(52) U.S. Cl. 424/443; 264/172.11

(57) ABSTRACT

A two component interpenetrating polymer network wound dressing material is formed of a biocompatible, hydrophilic first component such as gelatin or polyvinyl alcohol and a biocompatible elastomer such as HydroThane (trademark) by dissolving the components in a common solvent, initiating cross-linking of at least one of the components in the solution, and forming a film, fiber, bead or mesh from the solution.

This application claims the benefit of U.S. Provisional Application No. 60/427,933, filed Nov. 21, 2002.

### BACKGROUND OF THE INVENTION

1. Field of the Invention

This invention relates to an interpenetrating polymer network and to a method of producing the interpenetrating polymer network.

In particular, the invention relates to a hydrogel-elastomer interpenetrating polymer network (IPN) for use as a wound dressing. Interpenetrating polymer networks (IPNs) are multicomponent materials consisting of two or more cross-linked networks that are at least partially interlaced on a molecular scale but not covalently bonded to each other, and cannot be separated unless chemical bonds are broken. IPNs are distinguishable from blends, block copolymers and graft copolymers by (1) their ability to swell but not dissolve in solvents, and (2) suppression of their creep and flow. The preferred components of the IPN are (1) a hydrophilic biopolymer such as gelatin, chitosan, alginate or oxidized cellulose or a synthetic hydrogel such as polyvinyl alcohol, and (2) an elastomer such as a modified polyurethane. The IPN can be in the form of a film, fiber, sponge or mesh.

2. Discussion of the Prior Art

The inventors efforts were directed to the preparation of a wound dressing pad, many of which are described in the patent literature. Typical wound dressings include cotton gauze, coated nylon or polyethylene mesh. Fibers used in wound dressings include alginate, keratin and silver impregnated polyamide fibers. U.S. Pat. No. 5,676,967 discloses an aqueous combination of collagen and oligosaccharide coating on the surface of a polyethylene mesh. The mesh is used in a single layer to cover ulcers and burns. U.S. Pat. No. 6,123,958 discloses a non-reinforced, apertured gel web prepared from a water-soluble polysaccharide or cellulosic-polymer for treating burns. U.S. Pat. No. 5,961,478 relates to a super absorbent fiber consisting of polyacrylonitrile for use in wound dressings. Sorbsan (trademark) dressings (Pharma-Plast Ltd., Steriseal Division) are made of calcium alginate fibers with a non-woven structure, which maximizes absorption of wound exudate. The fibers of Sorbsan swell to form a soft, amorphous sodium-calcium alginate gel. Sorbsan is made from the calcium salt of alginic acid, prepared as a textile fiber, and presented as a loose ‘rope’ or packing for cavities, a ribbon for narrow wounds or sinuses, and a flat non-woven pad for application to larger open wounds. When in contact with serum, wound exudate or solutions containing sodium ions, the insoluble calcium alginate is partially converted to the soluble sodium salt, and a hydrophilic gel is produced, which overlays the wound and provides a micro-environment that is believed to facilitate wound healing. Sorbsan is indicated for moderate to high levels of exudages.

Fibracol (trademark) available from Johnson & Johnson Medical, Inc. is a 90% collagen-10% alginate wound dressing which combines the structural support of collagen and the gel forming properties of alginate into a soft and absorbent wound dressing.

In spite of the advances described above, there are certain significant aspects of wound dressings that do not appear to have been dealt with effectively. Deficiencies of some existing products include inadequate permeability to the outward passage of vapor from dressed wound sites, low absorption capacity, low hemostatic properties and a strong tendency to adhere to the biological elements of wounds during healing. This last factor involving attachment of wound dressings at a wound site results in damage to healing tissue during removal of dressings, thus prolonging overall healing.

Efforts to reduce such damage, e.g. by soaking off the attached material may have undesirable effects on biological healing elements involved with a Wound. Other important aspects of such a situation are the pain and adverse psychological effects that such experiences produce. Another area of concern is that of deep wounds involving internal organs such as intestines, liver, spleen and lungs. When organs are damaged and hemorrhaging, the current medical treatment frequently involves packing the injured organ or the abdominal cavity with gauze to diminish and control bleeding. The gauze is usually coarse and can cause irritation and bruising, while also becoming attached to the wound.

An object of the present invention is to provide a solution to the problems inherent to many existing wound dressings in the form of an IPN which has low adhesion to biological tissue.

### GENERAL DESCRIPTION OF THE INVENTION

According to one aspect, the invention relates to method of producing an interpenetrating polymer network comprising the steps of:

dissolving a biocompatible, hydrophilic first component selected from the group consisting of a biopolymer, a synthetic polymer and monomers and prepolymers of said biopolymer and synthetic polymer, and a second component selected from the group consisting of a biocompatible elastomer and monomers and prepolymers thereof in a common solvent to form a solution;

initiating cross-linking of at least one of the components; and

forming a film, fiber, bead or mesh from the solution.

According to another aspect, the invention relates to n interpenetrating polymer network comprising a biocompatible, hydrophilic first component selected from the group consisting of a biopolymer and a synthetic polymer; and a biocompatible elastomer as a second component, at least one of said first and second components being cross-linked.

As mentioned above, the hydrogel-elastomer IPN is produced by dissolving the two components in a common solvent, i.e. a solvent capable of dissolving both components, with subsequent mixing and cross-linking of the hydrophilic polymer and/or elastomer.

### DESCRIPTION OF THE PREFERRED EMBODIMENT

The hydrophilic first component is selected from the group consisting of polyvinyl alcohol, polyhydroxymethacrylate, polyethylene oxides, acrylamides, hydrophobically modified hydrogels, collagen, gelatin, fibronectin, cellulose, hydroxypropyl cellulose, hydroxypropylmethyl cellulose, methyl cellulose, ethyl, cellulose, carboxymethyl cellulose, carboxyethyl cellulose, a modified gelatin, alginate and oxidized cellulose, the preferred component being gelatin or a modified gelatin, specifically methacrylated gelatin. This material is hydrophilic, absorbent, biocompatible and possesses known hemostatic properties. Thus, the incorporation of gelatin into a wound dressing for application to hemmorrhagic living tissues would be expected to promote rapid hemostasis.

The hydrophobic second component include siloxane polymers such as polydimethylsiloxanes or vinyl containing siloxanes or polymethylhydrosiloxanes, polyethylene-vinylacetate (EVA), polytetramethylene oxide (PTMO), and HydroThane™. HydroThane is a trademark of Cardiotech International Inc. of Woburn, Mass. for a superabsorbent, thermoplastic modified thermoplastic elastomer. The particular product used in the present case is identified as HydroThane AR25-80A.

Common solvents for the two polymer components along with co-solvents, emulsifiers and smaller molecular weight polymers are used to increase the solubility of the two components in a compatible solvent to a functional level. One of the more important aspects of preparing hydrogel-elastomer IPN is finding a common solvent for the two components. When gelatin is used as the biopolymer component of the IPN, suitable solvents include glycerol, water, trifluoroethane and acetic acid. N-methylformamide, dimethylsulfoxide, formamide, acetamide, thioacetamide, propionamide, 2-pyrrolidinone, N-ethylurea, urea and thiourea derivatives also dissolve gelatin.

A co-solvent may be used to dissolve the first and second component in the common solvent. Suitable co-solvents include organic, nonpolar solvents such as cyclohexane, chloroform, benzene, toluene, methylene chloride, chlorobenze, chlorotoluene, methyl ethyl ketone, cyclic aromatics and halogenated cyclic aromatics, dimethylacetamide or N-methylpyrollidone.

Drugs or active ingredients may be introduced into the solution at this point providing that the drug or active ingredient is not adversely affected by the solvents or temperatures used to prepare the materials.

Ideally, the cross-linking reaction for the preparation of IPNs should be fast so that crosslinks are formed before phase separation begins to occur. The crosslink reaction rate may be increased by elevating the temperature or concentrations of the reagents. Once the cross-linking reaction has taken place the IPN may be washed for up to two weeks with water or solvent to fully remove all reagents and unreacted polymer materials.

During the preparation of IPN fibers consisting of gelatin-elastomer, cross-linking of the gelatin component may also only be effected once the fibers have been formed. Fibers may be formed individually using apparatus similar to a hypodermic needle where the solution is loaded into the barrel and the plunger is depressed at a slow rate to form a fiber. Heat or UV light may be used to cross-link the polymer components as the fiber is formed.

Drugs may be incorporated into the IPN via dispersion, dissolution, adsorption or chemical linkage depending upon the method used to combine the two polymers as well as the solubility properties of the drug. In the case of a gelatin-HydroThane film, drugs may be dissolved or dispersed in the gelatin-HydroThane reaction mixture prior to cross-linking of the gelatin or a solution of the drug can be adsorbed into the finished IPN material.

The IPN is formed into a film, fiber, sponge (open cell structure) or a mesh for use in a wound dressing.

The following examples further illustrate the invention.

### EXAMPLE 1

Methacrylation of Gelatin

10 g of gelatin was added to 100 mL of phosphate buffered saline (PBS, pH 7.4) and the mixture was stirred at 50° C. until complete dissolution. A 0.5 mL aliquot of 94% methacrylic anhydride was added to the gelatin solution. The reaction mixture was stirred for 60 min at approximately 50° C., and dialysed against distilled water at room temperature for one week before freeze-drying for 4 to 6 days. The dialysis membranes that were used had a molecular weight cut-off of 12000-14000.

Preparation of Methacrylated Gelatin Solution in DMSO

A 7.5 wt % methacrylated gelatin soluton was prepared in DMSO and immediately used for preparation of an interpenetrating polymer network (IPN). Alternately, an 18 wt % methacrylated gelatin solution can be prepared in DMSO and left at room temperature in a sealed scintillation vial (i.e., no nitrogen protection) for a lengthy period until it is diluted to 7.5 wt % in DMSO for IPN preparation.

Preparation of Gelatin HydroThane IPN

A 0.67 g sample of 7.5 wt % methacrylated gelatin in DMSO was mixed with 1.25 g of 4 wt % HydroThane in DMSO in a scintillation vial. A 91 μl aliquot of 10 wt % 2,2-dimethoxy-2-phenylacetophenone (available from Ciba Specialty Chemicals Canada of Toronto, Ontario under the trademark Irgacure 651) in DMSO was then added. The mixture was vigorously vortexed for about 30 s, and purged with nitrogen for 5 minutes in the scintillation vial. The mixture in the vial was UV-irradiated for 15 min at 350 nm at an intensity of 9 m W/cm² to form an IPN film. The resulting film was washed in a 0.1% sodium azide aqueous solution to remove all residual DMSO.

### EXAMPLE 2

Methacrylation of Polyvinyl Alcohol

5 g of polyvinyl alcohol (PVA) was added to 45 g of DMSO, and the resulting mixture was stirred at 80° C. until complete dissolution. A 0.5 mL aliquot of 94% methacrylic anhydride was added to the PVA solution. The reaction mixture was stirred for 60 min at approximately 50° C., and then precipitated into acetone. The precipitant was dialysed against distilled water at room temperature for one week before freeze-drying for 4 to 6 days. The dialysis membranes that were used had a molecular weight cut-off of 12000-14000. A 7.5 wt % methacrylated PVA solution was prepared in DMSO and immediately used for preparation of an interpenetrating polymer network (IPN).

Preparation of PVA-HydroThane IPN

A 0.67 g sample of 7.5 wt % methacrylated PVA in DMSO was mixed with 1.25 g of 4 wt % HydroThane in DMSO in a scintillation vial. A 91 μl aliquot of 10 wt % Irgacure 651™ in DMSO was then added. The mixture was vigorously vortexed for about 30 s, and purged with nitrogen for 5 minutes in the scintillation vial. The mixture in the vial was UV-irradiated for 15 min at 350 nm at an intensity of 9 mW/cm² to form an IPN film. The resulting film was washed in a 0.1% sodium azide aqueous solution to remove all residual DMSO.

The preferred form of the IPN of the present invention is a three-dimensional open mesh which is resilient, i.e. compressible and expansible. Such as mesh can be used to advantage in a wound having a small opening and a large area of internal tissue damage. When inserted into the wound, the mesh expands to fill the cavity in the body.

1. A method of producing an interpenetrating polymer network comprising the steps of:
dissolving a biocompatible, hydrophilic first component selected from the group consisting of a biopolymer, a synthetic polymer and monomers and prepolymers of said biopolymer and synthetic polymer, and a second component selected from the group consisting of a biocompatible elastomer and monomers and prepolymers thereof in a common solvent to form a solution;
initiating cross-linking of at least one of the components; and
forming a film, fiber, bead or mesh from the solution.

2. The method of claim 1, wherein a hydrophilic polymer and a an elastomer selected from the group consisting of silicone, polyurethane and a modified polyurethane are dissolved in a common solvent to form a solution; cross-linking of at least one of the components is initiated, the resulting resin solution is shaped to form a film or fiber.

3. The method of claim 2, wherein the resin solution is shaped to form a three-dimensional open mesh.

4. The method of claim 2, wherein the first component is selected from the group consisting of a polyvinyl alcohol, polyhydroxymethacrylate, polyethylene oxides, acrylamides, hydrophobically modified hydrogels, collagen, gelatin, fibronectin, cellulose, hydroxypropyl cellulose, hydroxypropylmethyl cellulose, methyl cellulose, ethyl cellulose, carboxymethyl cellulose, carboxyethyl cellulose, modified gelatin, alginate and oxidized cellulose, and the second component is selected from the group consisting of polyurethane-polydimethylsiloxane copolymers, vinyl containing siloxanes, polymethylhydrosiloxanes, polyethylene-polyvinylacetate, polypropylene oxide, polytetramethylene oxide, polytetrafluoroethylene, polystyrene and HydroThane.

5. The method of claim 1, wherein the first component is gelatin and the second component is HydroThane.

6. The method of claim 1, wherein gelatin is subjected to methacrylation to produce methacrylated gelatin; the methacrylated gelatin and HydroThane are dissolved in a common solvent to form a solution; and the solution is UV-irradiated to effect cross-linking.

7. The method of claim 6, wherein the common solvent is dimethyl sulfoxide.

8. The method of claim 7, wherein methacrylation is effected with methacrylic anhydride.

9. The method of claim 1, wherein the first component is polyvinyl alcohol and the second component is HydroThane.

10. The method of claim 9, wherein the polyvinyl alcohol is subjected to methacrylation to produce methacrylated polyvinyl alcohol; the methacrylated polyvinyl alcohol and HydroThane are dissolved in a common solvent to form a solution; and the solution is UV-irradiated to effect cross-linking.

11. An interpenetrating polymer network comprising a biocompatible, hydrophilic first component selected from the group consisting of a biopolymer and a synthetic polymer; and a biocompatible elastomer as a second component, at least one of said first and second components being cross-linked.

12. The interpenetrating polymer network of claim 11 in the form of a three-dimensional, resilient, open mesh.

13. The interpenetrating polymer network of claim 11, wherein said first component is selected from the group consisting of polyvinyl alcohol, polyhydroxymethacrylate, polyethylene oxides, acrylamides, hydrophobically modified hydrogels, collagen, gelatin, fibronectin, cellulose, hydroxypropyl cellulose, hydroxypropylmethyl cellulose, methyl cellulose, ethyl cellulose, carboxymethyl cellulose, carboxyethyl cellulose, a modified gelatin, alginate and oxidized cellulose, and said second component is selected from the group consisting of vinyl containing siloxanes, polymethylhydrosiloxanes, polyethylene-polyvinylacetate, polytetramethylene oxide and HydroThane.

14. The interpenetrating polymer network of claim 13, wherein the first component is gelatin and the second component is HydroThane.

15. The interpenetrating polymer network of claim 13, wherein the first component is methacrylated gelatin and the second component is HydroThane.

16. The interpenetrating polymer network of claim 13, wherein the first component is polyvinyl alcohol and the second component is HydroThane.

17. The interpenetrating polymer network of claim 13, wherein the first component is methacrylated polyvinyl alcohol and the second component is HydroThane.
